(19) United States
(12) Patent Application Publication
(10) Pub. No.: US 2005/0090466 A1
(43) Pub. Date: Apr. 28, 2005

(54) Inhibitors of PACE4 for the treatment of arthritis

(75) Inventors: Anne-Maria Malfait, St. Louis, MO (US); Micky Tortorella, O'Fallon, MO (US)

Correspondence Address: PHARMACIA CORPORATION;GLOBAL PATENT DEPARTMENT, POST OFFICE BOX 1027, ST. LOUIS, MO, 63006, US

(73) Assignee: Pharmacia & Upjohn Company LLC

(21) Appl. No.: 10/942,581

(22) Filed: Sep. 16, 2004

Related U.S. Application Data
(60) Provisional application No. 60/503,196, filed on Sep. 16, 2003.

(51) Int. Cl. A61K 48/00; A61K 39/395

(52) U.S. Cl. 514/44; 424/146.1

(57) ABSTRACT

Disclosed are methods and compositions for the treatment of arthritis using inhibitors of PACE4.

This application claims priority to application 60/503,196 filed Sep. 16, 2003.

### FIELD OF THE INVENTION

The present invention relates to methods of treating and prevention osteoarthritis, and more particularly, methods of inhibiting proprotein convertases responsible for processing precursor enzymes that degrade components of cartilage.

### BACKGROUND OF THE INVENTION

Degradation of articular cartilage, resulting in the loss of its biomechanical properties, is the hallmark of osteoarthritis (OA). The primary cause of this process is elevated levels of proteolytic enzymes that degrade cartilage aggrecan and type II collagen. Aggrecan loss, which is an early and perhaps the most critical event in the progression of arthritis, can be ascribed to increased activity of aggrecanases that cleave the core protein. Two cartilage aggrecanases, aggrecanase-1 and aggrecanase-2, have been identified. They are zinc metalloproteinases belonging to the a disintegrin and metalloproteinase with thrombospondin motifs (ADAMTS) family, and are designated ADAMTS-4 and ADAMTS-5, respectively. Both are synthesized by chondrocytes in a latent, inactive form, thus requiring activation before they exert their activities against aggrecan.

Proprotein convertases (PC) are serine proteases whose major function is the proteolytic processing of precursor proteins into their functionally active forms through cleavage at the C-terminus of the consensus sequence RXXR. PC's are intracellular enzymes found in the cytosol, transgolgi membrane, cellular vesicles and the cell membrane. Some PC's are membrane bound, while others are free. A subgroup of proprotein convertases that cleave precursor proteins at a pair of basic amino acid residues within the precursor protein are called PACE, an acronym for paired-basic amino acid cleaving enzymes. One member of the PACE family of proprotein convertase enzymes is known as PACE4 (SEQ ID NO. 1).

Several inhibitors of PACE4 are known, such as polyarginine peptides and the chloromethylketone peptide inhibitor RVKR-CMK. Unlike the homolog PC PACE, PACE4 is not significantly inhibited by the mutant serine protease inhibitor (serpin) α1 antitrypsin Pittsburgh (α1-ATp), and equivocally inhibited by the mutant α1 antitrypsin Portland (α1-PDX). Inhibitors of PACE4 gene expression are also-known, such as hASH-1 and MASH-1. To date, few PACE4 substrates well characterized, the most notable being vonWillibrand factor.

### SUMMARY OF THE INVENTION

It has now been discovered that PACE4 is responsible, at least in part, for the processing and activation of aggrecanase-1 (ADAMTS-4) and aggrecanase-2 (ADAMTS-5). PACE4 is secreted by articular chondrocytes into the extracellular matrix of OA cartilage resulting in the activation of ADAMTS-4 and ADAMTS-5 and subsequent aggrecan degradation. Aggrecanase-1 is believed to be responsible, at least in part, for the degradation of cartilage in arthritis conditions, especially in osteoarthritis. Therefore, an embodiment of the present invention is directed to a method of preventing or treating an arthritis condition by inhibition of PACE4 in a subject in need thereof. Another embodiment of the present invention is directed to compounds and compositions for blocking PACE4. A further embodiment of the present invention is directed to pharmaceutical compositions for the treatment of arthritis comprising a blocker of PACE4.

Further scope of the applicability of the present invention will become apparent from the detailed description provided below. However, it should be understood that the following detailed description and examples, while indicating some embodiments of the invention, are given by way of illustration only since various changes and modifications within the spirit and scope of the invention will become apparent to those skilled in the art from this detailed description.

### BRIEF DESCRIPTION OF THE DRAWINGS

FIG. 1 is a bar graph showing the percent decrease in aggrecanase activity from TNF-α and OSM stimulated chondrocyte controls in condrocytes with the addition of siRNA of SEQ ID NO. 2 and SEQ ID NO. 3;

FIG. 2 depicts an electrophoresis gel showing aggrecan-degradation products in untreated (lane 1), TNF/OSM treated control (lane 2) and TNF/OSM treated, PACE4 siRNA treated cartilage explants; and

FIG. 3 is a bar graph showing glycosaminoglycan (GAG) release from cartilage explants into the supernatant is reduced in TNF/OSM treated cartilage explants with the addition of siRNA of SEQ ID NO. 2 and SEQ ID NO. 3 (narrow shaded bar) as compared with normal control (unshaded bar) and TNF/OSM treated cartilage explants with nonsense dsRNA.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery. The contents of each of the references cited herein, including the contents of the references cited within these primary references, are herein incorporated by reference in their entirety.

### Definitions

The term “PACE4,” as used herein, means the dibasic proprotein convertase enzyme with the SWISSPROT accession number P29122, SEQ ID NO. 1, Chemical Abstracts Registry Number: 151662-24-7, described in U.S. Pat. No. 5,863,756 issued to Barr et al., herein incorporated by reference. PACE4 is also known as protein convertase 6, Endoprotease PACE4; PACE4 proteinase; Paired basic amino acid cleaving enzyme 4; Precursor convertase PACE4; Propeptidase PACE4; Proprotein convertase PACE4; and Proprotein convertase SPC4.

The term “aggrecanase,” as used herein, and unless otherwise qualified, means either the enzyme aggrecanase-1 (also known as ADAMTS-4), and aggrecanase-2 (also known as ADAMTS-5).

The terms “latent aggrecanase, precursor aggrecanase, immature aggrecanase, or pre-aggrecanase,” as used interchangeably herein, means the unprocessed form of aggrecanase, that is to say, the form of aggrecanase prior to processing by a proprotein convertase, particularly PACE4. This form of aggrecanase is not enzymatically active, that is, not functional to cleave aggrecan.

The terms “active aggrecanase, functional aggrecanase, mature aggrecanase, or processed aggrecanase,” as used interchangeably herein, means the form of aggrecanase that is enzymatically active, that is, functional to cleave aggrecan.

The term “α1-PDX,” as used herein, means alpha1-antitrypsin variant Portland, an engineered serpin (that is, serine protease inhibitor) that contains the minimal SPC consensus motif in its reactive loop.

The term “RVKR-CMK,” as used herein, means the irreversible chloromethylketone peptide inhibitor, Decanoyl-Arg—Val—Lys—Arg-chloromethylketone, a broad PC inhibitor. RVKR-CMK has the following structure:

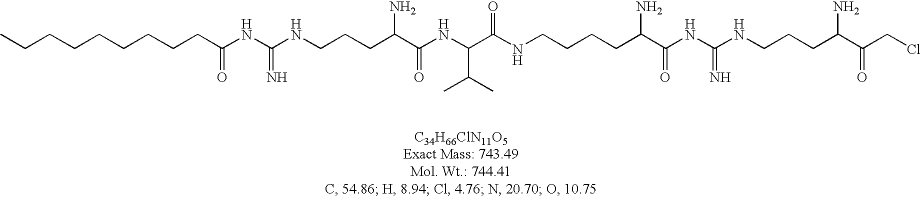

The term “hASH-1,” as used herein, means human achaete-scute homologue 1. It is believed that hASH-1 down-regulates PACE-4 gene expression.

The term “MASH-1,” as used herein, means mammalian achaete-scute homologue 1, a mammalian homologue of the Drosophila achaete-scute complex. It is believed that MASH-1 down-regulates PACE-4 gene expression.

The term “RNA,” as used herein, means ribonucleic acid.

The term “mRNA,” as used herein, means messenger RNA.

The term “target mRNA,” as used herein, means a predetermined mRNA selected for direct or indirect manipulation, modification or inhibition.

The term “target peptide,” as used herein, means a predetermined peptide selected for direct or indirect manipulation, modification or inhibition.

The term “asRNA,” as used herein, means anti-sense RNA.

The term “siRNA,” as used herein, means small interfering RNA, or short interfering RNA. siRNA's are generally double stranded, and are about nineteen to about twenty-five base pairs in length.

The term “RNAi,” as used herein, means RNA interference, a process whereby target mRNA is selectively degraded by a RISC, thereby reducing or eliminating expression of a target peptide.

The term “RISC,” as used herein, means an RNA Induced Silencing Complex.

A pharmaceutically acceptable carrier includes, but is not limited to, physiological saline, Ringer's, phosphatesolution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective

The term “pharmacologically effective amount” means that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the compositions of the invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group Ia) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

“Effective amount” means the dose or effective amount to be administered to a patient and the frequency of administration to the subject which is readily determined by one or ordinary skill in the art, by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

“Co-administration” and “co-administered” mean both taken in a single delivery vehicle, taken together contemporaneously, or taken within a period of time sufficient to receive a beneficial effect from both of the constituent agents of the combination.

The term “subject” for purposes of treatment includes any human or animal subject who has any one of the known arthritis disorders, and is preferably a human subject. For methods of prevention, the subject is any human or animal subject, and preferably is a human subject who is at risk for obtaining arthritis. The subject may be at risk due to genetic predisposition, injury, age and the like.

The term “treatment,” as used herein, unless otherwise qualified, means prophylactic, palliative, restorative or curative treatment.

The term “prophylactic treatment,” as used herein, means preventative treatment for a subject predisposed to a PACE4 mediated condition. The predisposition may be due to genetic factors, age, sex, injury, and the like.

The term “palliative treatment,” as used herein, means treatment with the objective of relieving symptoms of a condition, without significantly mitigating or eliminating the underlying condition.

The term “restorative treatment,” as used herein, means treatment effective to mitigate the underlying condition.

The term “curative treatment,” as used herein, means treatment effective to cause the complete remission of the underlying condition.

The term “arthritis,” as used herein, and unless otherwise qualified, includes without limitation rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis.

The singular indefinite articles “a” and “an,” when used in a Markush group, are intended to include the plural.

Both ADAMTS-4 and ADAMTS-5 have a PC cleavage site, RAKR²¹² (SEQ ID NO. 5 ) and RRRR²⁶¹ (SEQ ID NO. 6 ) (Arg—Ala—Lys—Arg²¹² and Arg—Arg—Arg—Arg²⁶¹) respectively, located within their pro-domain downstream from the cysteine switch, suggesting that one or more PC's activate these proteinases. Addition of several recombinant PC's, including furin, PC5/6, PC7 and PACE4, to unstimulated live or dead bovine or human cartilage explants triggered aggrecan catabolism, suggesting activation of constitutively present latent aggrecanases. Furthermore, IL-1-induced aggrecan breakdown could be blocked with the irreversible chloromethylketone peptide inhibitor, RVKR-CMK (Decanoyl-Arg—Val—Lys—Arg-chloromethylketone), a broad PC inhibitor, but not with the potent furin inhibitor alpha1-PDX. Endogenous PC activity was detected in the extracellular matrix of IL-1-stimulated bovine cartilage and human OA cartilage, but not in that of normal cartilage, suggesting that a PC is secreted from chondrocytes in pathological conditions. The endogenous PC activity was purified from the extracellular matrix of OA cartilage using conventional and affinity chromatography. The enzymatic profile of this activity was found to be identical to that of PACE4, and its identity was confirmed to be PACE4 by immunoprecipitation. Recombinant PACE4 was shown to activate recombinant proADAMTS-4 and proADAMTS-5 in what appears to be a 2-step activation requiring cleavage at the PC cleavage site located within the N-terminal domain and at another PC cleavage site located within the C-terminal thrombospondin domain at RRTR⁵⁶⁵ (SEQ ID NO. 7) (Arg—Arg—Thr—Arg⁵⁶⁵) and RAIYR⁶¹⁴ (SEQ ID NO. 8) (Arg—Ala—Ile—Tyr—Arg⁶¹⁴) of ADAMTS-4 and ADAMTS-5, respectively. Finally, evaluation of human OA cartilage by immunohistochemistry demonstrated that PACE4 is co-localized with ADAMTS-4 protein, with the aggrecanase-generated aggrecan neoepitope, NITEGE³⁷³ (SEQ ID NO. 9) (Asn—Ile—Thr—Glu—Gly—Glu³⁷³), and with areas of aggrecan depletion.

Inhibition of PACE4 may be accomplished by reducing or halting expression of the PACE4 gene. The bHLH transcription factors Hash-1, Hash-2, Mash-1 and Mash-2 are known to inhibit expression of PACE4. Therefore, in one embodiment of the present invention, a therapeutically effective amount of a transcription factor selected from Hash-1, Hash-2, Mash-1 and Mash-2 is administered to a subject suffering from arthritis, to prevent expression of PACE4 and subsequent processing of aggrecanase.

Inhibition of PACE4 may in addition be accomplished by administering an antibody that is specific for and capable of inactivating PACE4. In one embodiment of the present invention, a monoclonal antibody that is specific for PACE4 and is capable of inactivating PACE4 is administered to a subject in need thereof. In another embodiment, one or more polyclonal antibodies that are specific for PACE4 and capable of inactivating PACE4 are administered to a subject in need thereof.

Antibodies can be raised, using well known techniques, to a portion of the PACE4 enzyme such that activity of the PACE4 enzyme is disrupted or abolished. For example, an antibody raised to the artificial sequence Arg—Met—Leu—Asp—Gly—Asp—Val—Thr—Asp (SEQ ID NO. 4) located in the catalytic domain of mature PACE4, should abolish enzymatic activity of PACE4. Such an antibody could be raised by introducing the sequence, together with an adjuvant, for example, into an animal or human cell line capable of producing antibodies. For example, a keyhole limpet hemocyanin may be attached to the peptide by an appropriate linker, and introduced into an animal or human cell line to provoke an immune response, thereby raising antibodies to the target peptide sequence, such as SEQ ID NO. 4. Under appropriate circumstances, the antibody may be “humanized,” so as to prevent an undesirable immune response to the anti-PACE4 antibody itself, when the anti-PACE4 antibody is used as a treatment in humans for inhibiting PACE4. The antibody may be directly introduced into the joint by means of injection, for example, to increase penetration into the synovium and cartilage.

PACE4 antibodies useful in the present invention include monoclonal, chimeric, humanized, resurfaced, and recombinant antibodies and fragments thereof which are characterized by high affinity binding to TNF and low toxicity. In particular, an antibody where the individual components such as the variable region, constant region and framework, individually and/or collectively possess low immunogenicity is useful in the present invention. The antibodies which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

The antibody also includes a fragment or a derivative of such an antibody, such as one or more portions of the antibody chain, such as the heavy chain constant or variable regions, or the light chain constant or variable regions. Fragments lack the Fc fragment of intact antibody, clear more rapidly from the circulation, and can have less non-specific tissue binding than an intact antibody.

Chimeric antibodies are immunoglobulin molecules characterized by two or more segments or portions derived from different animal species. Chimeric antibodies include monovalent, divalent, or polyvalent immunoglobulins. Antibodies comprise individual heavy (H) and/or light(L) immunoglobulin chains. A chimeric H chain comprises an antigen binding region derived from the H chain of a non-human antibody specific for PACE4, which is linked to at least a portion of a human H chain C region (CH), such as CHI or CH2. A chimeric L chain comprises an antigen binding region derived from the L chain of a non-human antibody specific for TNF, linked to at least a portion of a humam L chain C region (CL).

Another method of inhibiting PACE4 is by way of reducing expression of PACE4 at the level of mRNA. Interference with the expression of PACE4 may be accomplished, for example, using RNA interference (RNAi).

RNAi currently comprises three techniques—antisense RNA (asRNA), double-stranded RNA (dsRNA), and small, interfering RNA (siRNA)—all of which cause RNA interference (RNAi), effectively shutting down protein synthesis from the targeted gene. RNAi starts with the appearance, from any one of various sources, in the cytoplasm of a cell of a strand of RNA that is complementary (antisense) to an mRNA transcript (sense) that is being produced normally by the cell. Under the right conditions and at the right concentrations, the antisense and sense strands anneal to form double-stranded RNA. The double-stranded RNA is rapidly degraded by a type III endonuclease called DICER, which cleaves the long double strand into 19 to 23 basepair long fragments. These oligoribonucleotides then direct the degradation of the rest of the complementary transcripts though the RNA-induced Silencer Complex (RISC) or RNA-dependent RNA Polymerase (RdRP) or both—different reports from different organisms show slightly different mechanisms. The result is the ongoing degradation of mRNA matching that of the oligonucleotide (hence the name: small, interfering RNA's) almost as rapidly as the nucleus can produce the transcripts. Thus, the RNAi mechanism can be activated by adding antisense RNAs, pre-annealed double-stranded RNAs, or the siRNAs themselves: all result in the presence of siRNAs in the cell and the continuous degradation of complementary transcripts. Antisense transcripts are easy to induce transgenically, but don't always anneal to their sense counterparts at high enough levels to induce RNAi. ds RNA gives the best response, but is difficult to get into the cells at high levels, and in mammals causes a host defense response of high levels of interferon, which is often fatal to the affected cells. siRNAs are relativly easy to get into cells, but some siRNAs are better at knocking down transcripts than others, such that choosing the wrong siRNA can result in little or no interference. One approach at present is to use a pool of siRNAs for each gene in the hopes that at least one will give good repression. Another, rational approach for selection of appropriate dsRNA for siRNA is found in International Publication WO 2004045543, incorporated herein by reference.

### EXAMPLE

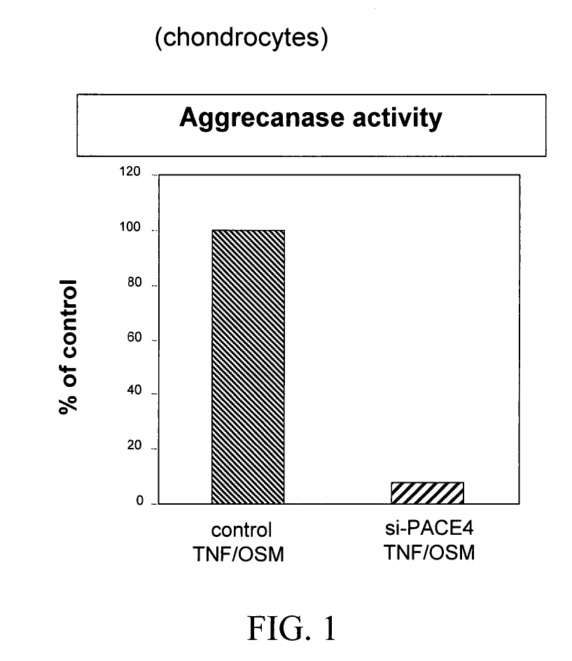

The sequence CGGCAAUGAUUAUGACCCAtt (SEQ ID NO. 2) was selected as a sense strand for dsRNA. The corresponding antisense strand, UGGGUCAUAAUCAUUGCCGtt, (SEQ ID NO. 3) was also selected. dsRNA was synthesized commercially (Ambion, Austin, Tex. 78744-1832 USA) and 100 nM was introduced into cultured human chondrocytes and human cartilage stimulated with TNF-α and Oncostatin M (OSM). It is known that stimulation of cartilage or chondrocytes with TNF-α and OSM will induce cartilage degradation and upregulate aggrecanase. Nonsense dsRNA, that is, dsRNA with the same amino acids, but scrambled in sequence, were introduced into cultured human chondrocytes and human cartilage explants as controls. The control groups are not expected to induce RNAi, since the dsRNA is not expected to produce an inhibitory effect on mRNA. FIG. 1 shows the percent decrease in aggrecanase activity from TNF-α and OSM stimulated chondrocyte controls in condrocytes with the addition of siRNA of SEQ ID NO. 2 and SEQ ID NO. 3. It can be seen that a greater than eighty percent reduction in aggrecanase activity was observed when PACE4 mRNA was inhibited.

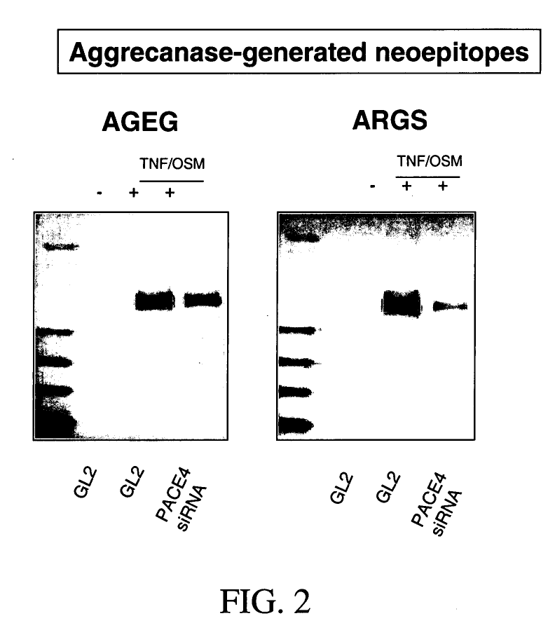

Referring now to FIG. 2, the aggrecanase mediated cleavage of aggrecan, shown by the aggrecan derived peptide neopitope fragment AGEG (SEQ ID NO. 10) and the aggrecan derived peptide neopepitope fragment ARGS (SEQ ID NO. 11) was reduced with interfering dsRNA, as compared to controls.

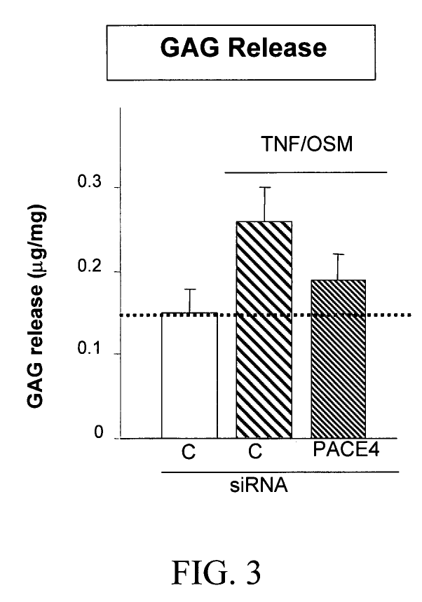

Glycosaminoglycan (GAG) release from proteoglycan and depressed proteoglycan synthesis are thought to be correlated with cartilage destruction in arthritis. Referring now to FIG. 3, glycosaminoglycan (GAG) release from cartilage explants into the supernatant is shown to be reduced in TNF/OSM treated cartilage explants with the addition of siRNA of SEQ ID NO. 2 and SEQ ID NO. 3 (narrow shaded bar) as compared with normal control (unshaded bar) and TNF/OSM treated cartilage explants with nonsense dsRNA.

### Pharmaceutical Compositions

A pharmaceutical composition of the present invention can take a wide variety of forms. For example, the composition can take the form of a tablet, a lozenge, a cachet, a capsule, a chewing gum, a chewable tablet, a controlled release formulation, a sustained-release formulation, a fast-dissolving film, a gel (e.g., a gel capsule), a semi-solid, a solution (aqueous or non-aqueous), a suspension, an intimate mixture of the components, a lyophilisate, or any combination of two or more of the above. Because of the physical and chemical nature of antibodies used in the present invention, an antibody is typically administered in the form of a solution, for example by injection. For example, a PACE4 antibody can be stored as the solution or, for example, in a solid form such as a lyophilisate.

In one embodiment of the present invention, the composition is a solid dosage form. For example, the solid dosage form can be an oral dosage form. In yet another embodiment, the oral dosage form is selected from a group consisting of a tablet, a capsule, a pill, a gel cap, and granules. In another embodiment the oral dosage form is a capsule.

In another embodiment the capsule is a time-release capsule. Such a time-release capsule can, for example, release the active ingredients from a matrix, or in another example it can release the active ingredients at different rates from a mixture of controlled release matrices.

In another embodiment, the oral dosage form is a tablet dosage form. In one embodiment, the tablet can comprise a single layer. In another embodiment the tablet dosage form can comprise, for example, a multiple layer tablet dosage form (for example, a separate layer for each active ingredient), a wafer, a sustained release tablet dosage form, a core-mantle tablet dosage form, and a side-by-side tablet dosage form (for example, a side for each active ingredient).

In another embodiment the tablet dosage form comprises a sustained release tablet dosage form. In yet another embodiment, the tablet dosage form comprises a core and mantle tablet dosage form.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound optimally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing an active ingredient may be formulated with sweetening agents, for example glycerol, sorbitol, or sucrose. Such formulations may also contain a demulcent, a preservative and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Also encompassed by the present invention is buccal or sub-lingual administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

Pharmaceutical compositions suitable for parenteral administration can conveniently comprise sterile aqueous preparations of a compound of the present invention. These preparations can be, for example, administered intravenously, although administration can also be effected by means of subcutaneous, intramuscular, or intradermal injection or by infusion. Such preparations can conveniently be prepared by admixing the compound (for example in the form of a solid such as a lyophilisate) with water and rendering the resulting solution sterile and isotonic with the blood. A frequently suitable sterile aqueous preparation can be prepared using Water for Injection. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Administration of a compound of the present invention can also be by inhalation, in the form of aerosols or solutions for nebulizers. Therefore, in one embodiment, the compound is administered by direct inhalation into the respiratory system of a subject for delivery as a mist or other aerosol or dry powder.

Pharmaceutical compositions suitable for topical application to the skin can, for example, take the form of an ointments, creams, lotions, pastes, gels, sprays, powders, jellies, collyriums, solutions or suspensions, aerosols, or oils.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

Carriers which can be used include petroleum jelly (e.g., Vaseline®), lanolin, polyethylene glycols, alcohols, and combinations of two or more thereof.

Solid dosage forms for the methods of the present invention, which include tablets, capsules, pills, and granules, which can be prepared with coatings and shells, such as enteric coatings and others well known in the art.

Compositions intended for oral use may be prepared according to any method known in the art for the manufacture of pharmaceutical compositions and such compositions may contain one or more agents selected from the group consisting of sweetening agents, flavoring agents, coloring agents, taste masking agents, and preserving agents in order to provide pharmaceutically useful and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc.

The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth, xanthan gum, and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above.

Additional excipients, for example sweetening, flavoring and coloring agents, may also be present.

The active ingredients may also be administered by injection as a composition wherein, for example, saline, dextrose, or water may be used as a suitable carrier.

Suitable inhalable formulations comprise the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Administration of the compositions of the present invention can also be rectally, for example, by way of a suppository. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

The compositions of the present invention can optionally be supplemented with additional agents such as, for example, viscosity enhancers, preservatives, surfactants and penetration enhancers.

Viscosity can be an important attribute of many medications. Drops that have a high viscosity tend to stay in the body for longer periods and thus, increase absorption of the active compounds by the target tissues or increase the retention time. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives are optionally employed to prevent microbial contamination during use. Suitable preservatives include polyquatemium-1, benzalkonium chloride, thimerosal, chlorobutanol, methyl paraben, propyl paraben, phenylethyl alcohol, edetate disodium, sorbic acid, or other agents known to those skilled in the art. The use of polyquatemium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g. Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

Pharmaceutically acceptable excipients and carriers encompass all the foregoing and the like. The above considerations concerning effective formulations and administration procedures are well known in the art and are described in standard textbooks.

Pharmaceutical compositions according to the present invention include those suitable for oral, rectal, topical, buccal (e.g., sublingual), and parenteral (e.g., subcutaneous, intramuscular, intradermal, intrathecal, intramedullary, or intravenous) administration, although the most suitable route in any given case will depend on the nature and severity of the condition being treated and on the nature of the particular compound which is being used. In some cases, the route of administration will be parenteral.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water, which render the resulting solution sterile and isotonic with the blood. Injectable compositions according to the invention will generally contain from 0.1 to 10% w/w of a compound disclosed herein.

Oral delivery of the compositions of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Administration may also be by transvaginal delivery through the use of an intravaginal device. Transvaginal delivery may be desirable for many subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular example, the compound or compounds can be delivered from the patch by electrotransport or iontophoresis.

It will be appreciated that the amount of the present composition required for use in the treatment or prevention of the conditions described herein will vary within wide limits and will be adjusted to the individual requirements in each particular case. In general, for administration to adults, an appropriate daily dosage is described herein, although the dosages that are identified herein may be exceeded if expedient. The daily dosage can be administered as a single dosage or in divided dosages.

A compound may be administered on a regimen of several times per day, for example 1 to 4 times per day, alternatively once or twice per day.

A formulation intended for the oral administration of humans may contain from 0.5 mg to 7 g of active agent compounded optionally with an appropriate and convenient amount of carrier material, which may vary from about 5 to about 95 percent of the total composition. It is understood that specific dose levels of the therapeutic agents or therapeutic approaches of the present invention for any particular patient depends upon a variety of factors including the activity of the specific compound employed, the age, body weight, general health, sex, and diet of the patient, the time of administration, the rate of excretion, the severity of the particular disease being treated, and the form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of pain or inflammation in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, for example, 10 μM, one will desire to administer an amount of the drug that is effective to provide about a 10 μM concentration in vivo. Determination of these parameters is well within the skill of the art.

Numerous variations will occur to those skilled in the art in light of the foregoing disclosure. Such variations are intended to fall within the scope of the appended claims.

1. A method for treating arthritis, in a subject in need of such treatment, comprising administering to the subject a treatment effective amount of a blocker of PACE4.

2. The method of claim 1 wherein said blocker of PACE4 is an inhibitor of PACE4 enzyme.

3. The method of claim 2 wherein said inhibitor of PACE4 enzyme is one or more compounds selected from the group consisting of: a monoclonal antibody specific to and capable of inactivating PACE4; one or more polyclonal antibodies specific to and capable of inactivating PACE5; a polyarginine compound; and the chloromethylketone peptide inhibitor RVKR-CMK.

4. The method of claim 1 wherein said blocker of PACE4 is an inhibitor of PACE4 gene expression.

5. The method of claim 4 wherein said inhibitor of PACE4 gene expression is one or two compounds selected from the group consisting of hASH-1 and MASH-1.

6. The method of claim 4 wherein said inhibitor of PACE4 gene expression is an inhibitor of RNA.

7. The method of claim 6 wherein said inhibitor of RNA is a dsRNA.

8. The method of claim 7 wherein said dsRNA has the sequence CGGCAAUGAUUAUGACCCAtt (SEQ ID NO. 2) as the sense strand, and UGGGUCAUAAUCAUUGCCGtt, (SEQ ID NO. 3) as the antisense strand.

9. The use of an inhibitor of PACE4 in the manufacture of a medicament for the treatment of arthritis.

10. A pharmaceutical composition comprising an arthritis treatment effective amount of a blocker of PACE4, together with a suitable, pharmaceutically acceptable carrier.

11. The pharmaceutical composition of claim 10 wherein the blocker of PACE4 is an inhibitor of PACE4 enzyme.

12. The pharmaceutical composition of claim 11 wherein said inhibitor of PACE4 enzyme is one or more compounds selected from the group consisting of: a monoclonal antibody specific to and capable of inactivating PACE4; a polyarginine compound, and the chloromethylketone peptide inhibitor RVKR-CMK.

13. The pharmaceutical composition of claim 10 wherein said blocker of PACE4 is an inhibitor of PACE4 gene expression.

14. The pharmaceutical composition of claim 13 wherein said inhibitor of PACE4 gene expression is one or two compounds selected from the group consisting of hASH-1 and MASH-1.

15. A method of preventing the activation of precursor aggrecanase into functionally active aggrecanase in a subject in need of aggrecanase inhibition comprising treating said subject with an effective amount of a blocker of PACE4.


SEQUENCE LISTING (11 sequences)

SEQ ID NO: 1 - protein, 969 aa, Homo sapiens
MPPRAPPAPGPRPPPRAAAATDTAAGAGGAGGAGGAGGPGFRPLAPRPWRWLLLLALPAACSAPPPRPVYTNHWAVQVLGGPAEADRVAAAHGYLNLGQIGNLEDYYHFYHSKTFKRSTLSSRGPHTFLRMDPQVKWLQQQEVKRRVKRQVRSDPQALYFNDPIWSNMWYLHCGDKNSRCRSEMNVQAAWKRGYTGKNVVVTILDDGIERNHPDLAPNYDSYASYDVNGNDYDPSPRYDASNENKHGTRCAGEVAASANNSYCIVGIAYNAKIGGIRMLDGDVTDVVEAKSLGIRPNYIDIYSASWGPDDDGKTVDGPGRLAKQAFEYGIKKGRQGLGSIFVWASGNGGREGDYCSCDGYTNSIYTISVSSATENGYKPWYLEECASTLATTYSSGAFYERKIVTTDLRQRCTDGHTGTSVSAPMVAGIIALALEANSQLTWRDVQHLLVKTSRPAHLKASDWKVNGAGHKVSHFYGFGLVDAEALVVEAKKWTAVPSQHMCVAASDKRPRSIPLVQVLRTTALTSACAEHSDQRVVYLEHVVVRTSISHPRRGDLQIYLVSPSGTKSQLLAKRLLDLSNEGFTNWEFMTVHCWGEKAEGQWTLEIQDLPSQVRNPEKQGKLKEWSLILYGTAEHPYHTFSAHQSRSRMLELSAPELEPPKAALSPSQVEVPEDEEDYTAQSTPGSANILQTSVCHPECGDKGCDGPNADQCLNCVHFSLGSVKTSRKCVSVCPLGYFGDTAARRCRRCHKGCETCSSRAATQCLSCRRGFYHHQEMNTCVTLCPAGFYADESQKNCLKCHPSCKKCVDEPEKCTVCKEGFSLARGSCIPDCEPGTYFDSELIRCGECHHTCGTCVGPGREECIHCAKNFHFHDWKCVPACGEGFYPEEMPGLPHKVCRRCDENCLSCAGSSRNCSRCKTGFTQLGTSCITNHTCSNADETFCEMVKSNRLCERKLFIQFCCRTCLLAG

SEQ ID NO: 2 - DNA, 21 bp, Artificial
  Sense strand of dsRNA for siRNA
cggcaaugauuaugacccatt

SEQ ID NO: 3 - DNA, 21 bp, Artificial
  Antisense dsRNA for siRNA
ugggucauaaucauugccgtt

SEQ ID NO: 4 - protein, 9 aa, Artificial
  artificial fragment of catalytic peptide domain of PACE4
RMLDGDVTD

SEQ ID NO: 5 - protein, 4 aa, Artificial
  Putative recognision site/cleavage site of human pro peptide or Aggrecanase
RAKR

SEQ ID NO: 6 - protein, 4 aa, Artificial
  Putative recognision/cleavage site of human pro Aggrecanase
RRRR

SEQ ID NO: 7 - protein, 4 aa, Artificial
  Putative cleavage site of human pro-ADAMTS-4
RRTR

SEQ ID NO: 8 - protein, 5 aa, Artificial
  Putative cleavage site for human pro ADAMTS-5
RAIYR

SEQ ID NO: 9 - protein, 6 aa, Artificial
  Aggrecanase generated neoepitope peptide of human aggrecan
NITEGE

SEQ ID NO: 10 - protein, 4 aa, Artificial
  aggrecanase mediiated aggrecan derived neopitope
AGEG

SEQ ID NO: 11 - protein, 4 aa, Artificial
  aggrecanase mediated aggrecan neoepitope
ARGS